(19) United States
(12) Patent Application Publication
(10) Pub. No.: US 2025/0059333 A1
(43) Pub. Date: Feb. 20, 2025

(54) BILAYER BIONIC DRUG-LOADED HYDROGEL, AND PREPARATION AND APPLICATION THEREOF

(71) Applicant: Chengdu Hengkui Biotechnology Co., Ltd., Chengdu (CN)

(72) Inventors: Wei ZHI, Chengdu (CN); Xin FANG, Chengdu (CN); Yuehao WU, Chengdu (CN); Jinjie WU, Chengdu (CN); Xiupeng ZHU, Chengdu (CN); Jianxin WANG, Chengdu (CN); Dong SUN, Chengdu (CN); Hongri WU, Chengdu (CN)

(21) Appl. No.: 18/937,934

(22) Filed: Nov. 5, 2024

Related U.S. Application Data
(63) Continuation-in-part of application No. 18/476,562, filed on Sep. 28, 2023, now abandoned.
(63) Continuation of application No. PCT/CN2023/102580, filed on Jun. 27, 2023.

(30) Foreign Application Priority Data
Jul. 11, 2022 (CN) 202210809586.0

(51) Int. Cl. C08J 3/075; A61L 26/00

(52) U.S. Cl. CPC C08J 3/075; A61L 26/0014; A61L 26/0023; A61L 26/0066; A61L 26/008; A61L 2300/406; C08J 2329/04; C08J 2405/08

(57) ABSTRACT

Disclosed is a bilayer bionic drug-loaded hydrogel, and preparation and application thereof. The hydrogel includes: an outer-layer hydrogel and an inner-layer hydrogel. The outer-layer hydrogel is prepared by: forming a polyvinyl alcohol hydrogel by directionally freezing a polyvinyl alcohol aqueous solution, soaking the polyvinyl alcohol aqueous solution in a sodium sulfate solution, and removing salt ions after soaking; and the inner-layer hydrogel is prepared by components of: a loaded drug, polyvinyl alcohol, chitosan, genipin, water, and a pH adjuster. The hydrogel of the present disclosure can be applied to deeply infected areas of wounds in open war wounds, and has protective, anti-inflammatory, haemostatic, reparative, anti-drug resistant bacterial effects, and other properties.

### CROSS-REFERENCE TO RELATED APPLICATIONS

This application is a continuation-in-part of U.S. patent application Ser. No. 18/476,562, filed on Sep. 28, 2023, now pending, which is a continuation of International Patent Application No. PCT/CN2023/102580 with a filing date of Jun. 27, 2023, designating the United States, and further claims the benefit of priority from Chinese Patent Application No. 202210809586.0, filed on Jul. 11, 2022. The content of the aforementioned application, including any intervening amendments thereto, is incorporated herein by reference in its entirety.

### TECHNICAL FIELD

The present disclosure belongs to the field of medical materials, and in particular, relates to a bilayer bionic drug-loaded hydrogel, and preparation and application thereof.

### BACKGROUND

Currently, wound dressings applied to wound care mainly include traditional wound dressings and modern wound dressings. The traditional wound dressings such as gauzes, bandages are generally used for dry and clean wounds. However, the traditional wound dressings fail to keep pathogenic bacteria out of wounds, leading to wound infections. Meanwhile, when the traditional wound dressings are used to care the wounds, the gauzes or bandages are probably adhered to the wounds, causing pain and secondary injuries to patients during dressing changing. Compared to the traditional wound dressings, the modern wound dressings can wet wound area, exchange air, absorb exudates, are not adhere to the wound surface, and promote autolytic debridement of the wound surface. As the modern dressings, hydrogel dressing has good biocompatibility, better water absorption, moisture retention, good air permeability and other advantages, and is thereby the hotspot in modern wound dressings field.

After years of studies and developments, various researchers have studied and developed different types of hydro gel wound dressings according to different wound types. These wound dressings are capable of wetting the wound surface, exchanging air, absorbing exudates, are not adhered to the wound surface, promoting autolytic debridement of the wound surface, and are antibacterial as well. However, those wound dressings are generally weak in tensile strength and toughness, and are not resistant to mechanical environment such as friction, stretching, and extrusion when applied to wound repair, and are prone to be damages, thus losing effects of protecting and repairing wound. As an important performance index for wound dressings, the antimicrobial effect is very important for the wound dressings. Currently, the antimicrobial effect of the hydrogel wound dressings is mainly achieved by the modifying material or loading antimicrobial drugs, while the antimicrobial property of the material itself is weak against strong infectious and drug-resistant pathogenic bacteria, thus resulting in undesirable anti-infection.

Polyvinyl alcohol (PVA) is a high-molecular polymer obtained by hydrolysis of vinyl acetate, and is widely used in the biomedical field because of its good water solubility, non-toxicity, non-irritation, and good biocompatibility, as well as the cross-linking ability by repeated freeze-thawing, radiation, and chemical reaction. Currently, PVA hydrogels with ultra-high and broadly modifiable mechanical strength can be prepared using the synergy of directional freezing and Hofmeister effect. However, such hydrogels have yet not been developed for skin wound dressings due to that the hydrogels prepared in this way have a dense and uniform structure, and mechanical properties would be severely impacted if other components are loaded, such as drugs or active factors. In addition, PVA does not have antibacterial and anti-inflammatory properties.

Chitosan is alkaline amino polysaccharide after deacetylation by chitin, and features sterilization, hemostasis, cell proliferation promotion, high biocompatibility and etc. Therefore, chitosan is widely used in the field of wound dressing. Genipin is a product of hydrolysis of Gardenia glycosides from Eucommia or Gardenia by β-glucosidase, and its molecules contain a large number of active groups, such as hydroxyl and carboxyl, which are extremely prone to react with compounds containing free amino groups (such as chitosan and gelatin). Therefore, genipin is often used as a cross-linking agent. As a natural cross-linking agent, toxicity of genipin is 10000 times lower than that of glutaraldehyde, a commonly used chemical cross-linking agent. Moreover, genipin also has anti-inflammatory and anti-oxidant effects. However, as a hydrogel, genipin has poor mechanical properties to resist the impact or friction from strong external forces. Therefore, when applied in dressing, genipin is prone to breakage and loss of function. In addition, the chitosan hydrogel is difficult to be firmly bonded to other hydrogels or materials upon gelling, showing poor adhesive property.

### SUMMARY

Various embodiments of the present disclosure are intended to provide a method of preparing a bilayer bionic drug-loaded hydrogel and application thereof, to solve the problem that existing wound dressings are generally weak in tensile strength and toughness and are not resistant to mechanical environment such as friction, stretching and extrusion when applied to wound repair. The hydrogel of the present disclosure can be applied to deeply infected areas in open war wounds, and has protective, anti-inflammatory, haemostatic, reparative, drug-resistant bacterial cleared effects and other properties.

The present disclosure provides a method for preparing a bilayer bionic drug-loaded hydrogel, comprising:
(S1) directionally freezing a polyvinyl alcohol aqueous solution, followed by soaking in a sodium sulfate solution and removal of salt ions to obtain an outer-layer hydrogel;
(S2) dissolving chitosan in water followed by addition of a pH adjuster to obtain a chitosan solution, and adding polyvinyl alcohol to the chitosan solution followed by heating under stirring to obtain a first mixture solution;
(S3) adding an antibacterial drug and genipin to the first mixture solution, followed by stirring in a dark environment to obtain a second mixture solution; and
(S4) spreading the second mixture solution on the outer-layer hydrogel, followed by freezing and thawing in a dark and clean environment to form an inner-layer hydrogel on the outer-layer hydrogel, such that the bilayer bionic drug-loaded hydrogel is obtained.

In an embodiment, in step (S1), a concentration of the sodium sulfate solution is 0.5-1.5 mol/L, and a weight percentage of the polyvinyl alcohol aqueous solution in the outer-layer hydrogel is 5-10%.

In an embodiment, the weight percentage of the polyvinyl alcohol aqueous solution in the outer-layer hydrogel is 5%.

In an embodiment, in step (S2), the pH adjuster is a weak acid.

In an embodiment, the pH adjuster is glacial acetic acid.

In an embodiment, in step (S2), a weight percentage of the polyvinyl alcohol in the first mixture solution is 5-10%, and a weight percentage of the chitosan in the first mixture solution is 2-4%.

In an embodiment, the chitosan and the polyvinyl alcohol are pre-sterilized by ultraviolet irradiation, and the water is pre-sterilized by autoclaving.

In an embodiment, in step (S2), the heating is performed at 85-95° C.

In an embodiment, in step (S3), the antibacterial drug is vancomycin, and a weight percentage of the genipin in the second mixture solution is 0.01-0.05%.

In an embodiment, in step (S4), the freezing and thawing is performed 1-5 times at −20-0° C., each lasting for 0.5-2 h.

In an embodiment, the inner-layer hydrogel has a double-network structure. The double-network structure includes a first network and a second network. The first network is a three-dimensional network structure formed by repeatedly freezing and thawing the polyvinyl alcohol, and the first network structure is a combination of hydrogen bonds between PVA molecular chains, microcrystals, and water in different bonding states at different scales. The second network is formed by chemically cross-linking molecules of the genipin and the chitosan.

In an embodiment, in step (S1), the freezing is performed at −80-0° C. for 0.5-4 h.

In summary, the method of preparing the bilayer bionic drug-loaded hydrogel of the present disclosure solves the problem that existing wound dressings are generally weak in tensile strength and toughness, and are not resistant to mechanical environment such as friction, stretching and extrusion when applied to wound repair, and has the following advantages:
1. According to the present disclosure, the secondary physical cross-linking (freezing and thawing) is used to form a strong molecular link between the outer-layer hydrogel and the inner-layer hydrogel through hydrogen bonding. The inner-layer hydrogel with a double-network structure is obtained through the synchronized physical (freezing) cross-linking/bio-cross-linking (genipin) reaction, which has a very different structure from that of the outer-layer hydrogel and is seamlessly connected to the outer-layer hydrogel.
2. According to the present disclosure, the outer-layer hydrogel is prepared using a directional freezing plus Hofmeister effect. The mechanical properties of the outer-layer hydrogel are regulated by controlling types and concentrations of salt ions, such that the mechanical properties of the hydrogel are fitted to the mechanical properties of skin.
3. According to the present disclosure, since the outer-layer hydrogel has mechanical properties similar to those of skin, and is capable of withstanding high intensity squeezing, pulling and rubbing, the wound surface of skin can be protected from negative external stimulation while the wound surface is kept dry and clean.
4. According to the present disclosure, the inner-layer hydrogel can seal the wound and be loaded with antimicrobial drugs or bioactive factors, which greatly improves the antimicrobial properties of the dressing, rendering the dressing a strong killing effect on strong pathogens such as Staphylococcus aureus, while enhancing the stability and repairing and regeneration function of wounds.
5. According to the present disclosure, genipin also acts to anti-inflammation and anti-oxidation while acting as a cross-linking agent. In addition, soft, moist and adhesive properties of the chitosan hydrogel are similar to those of subcutaneous tissue, thus the wound can be hermetically filled, such that the hydrogel is tightly bonded to the wound interface to effectively prevent re-infection and promote wound healing. The tight bond between the inner and outer layers mitigates dislocation of the inner and outer layers, thereby effectively providing simultaneous wound protection and regeneration.

### BRIEF DESCRIPTION OF THE DRAWINGS

FIG. 1a is a structural diagram of a bilayer drug-loaded hydrogel according to Example 1 of the present disclosure;

FIG. 1b is an optical photograph of a bilayer drug-loaded hydrogel according to Example 1 of the present disclosure;

FIG. 2a is a cross-sectional view of an outer-layer hydrogel observed by a scanning electron microscope according to Example 1 of the present disclosure;

FIG. 2b is a longitudinal cross-sectional view of an outer-layer hydrogel observed by a scanning electron microscope according to Example 1 of the present disclosure;

FIG. 3 is an image of an inner-layer hydrogel observed by a scanning electron microscope according to Example 1 of the present disclosure;

FIG. 4 is an image of a bilayer hydrogel observed by a scanning electron microscope according to Example 1 of the present disclosure;

FIG. 5a is a stress-strain curve of an outer-layer hydrogel in tension according to Example 1 of the present disclosure;

FIG. 5b is a relation diagram between Young's modulus of hydrogel and Young's modulus of skin (the largest circle area represents a Young's modulus range of the hydrogel) according to Example 1 of the present disclosure;

FIG. 6a shows mechanical properties of an outer hydrogel according to Example 1 of the present disclosure;

FIG. 6b shows mechanical properties of an inner hydrogel according to Example 1 of the present disclosure;

FIG. 7 shows a Fourier transform infrared spectroscopy (FTIR) of an inner-layer hydrogel according to Example 1 of the present disclosure;

FIG. 8 shows cytotoxicity evaluation of a bilayer hydrogel according to Example 1 of the present disclosure;

FIG. 9 shows an antioxygenic property (DPPH) of an inner-layer hydrogel according to Example 1 of the present disclosure;

FIG. 10a shows an antibacterial effect of a bilayer hydrogel on Escherichia coli according to Example 1 of the present disclosure;

FIG. 10b shows an antibacterial effect of a bilayer hydrogel on Staphylococcus epidermidis according to Example 1 of the present disclosure;

FIG. 10c shows an antibacterial effect of a bilayer hydrogel on Staphylococcus aureus according to Example 1 of the present disclosure;

FIG. 11a shows an adhesive property of a bilayer hydrogel to a hand according to Example 1 of the present disclosure;

FIG. 11b shows an adhesive property of a bilayer hydrogel to an elbow joint according to Example 1 of the present disclosure;

FIG. 11c shows an adhesive property of a bilayer hydrogel to a glass according to Example 1 of the present disclosure; and

FIG. 11d shows an adhesive property of a bilayer hydrogel to a plastic according to Example 1 of the present disclosure; and

FIG. 12 is an optical photograph of a drug-loaded hydrogel according to comparative example of the present disclosure.

### DETAILED DESCRIPTION

Technical solutions of the present disclosure will be described clearly and completely below. Obviously, the examples described are only some, rather than all examples of the present disclosure. Based on the examples of the present disclosure, all other examples obtained by those ordinary skilled in the art without creative efforts should fall within the scope of protection of the present disclosure.

### Example 1

Provided herein was a method for preparing a bilayer bionic drug-loaded hydrogel, which included the following steps.

### (1) Preparation of an Outer-Layer Polyvinyl Alcohol (PVA) Hydrogel

5 g of PVA was weighed and poured into a beaker, 95 mL of ultrapure (UP) water was added into the beaker, fully dissolved in a 90° C. water bath under heating and stirring, cooled and ultrasonically deformed, and a PVA solution with a mass fraction of 5% was obtained.

Liquid nitrogen was added into a directional freezing device, and when the temperature of the directional freezing device was constant, a mould containing the PVA solution prepared was placed on the directional freezing device for directional freezing at −60° C. for 1.5 h; after the PVA solution was fully frozen, the mould was removed from the directional freezing device and demoulding was carried out; the demoulded PVA hydrogel was soaked in a sodium sulfate solution for 72 h, followed by soaking in the UP water for 48 h, and the UP water was changed every 4 h to remove salt ions from the PVA hydrogel, and the outer-layer PVA hydrogel was obtained.

The mechanical properties of the outer-layer PVA hydrogel were adjusted by adjusting the concentration of PVA and the concentration of salting-out liquid.

### (2) Preparation of an Inner-Layer Hydrogel Precursor Solution

2 g of chitosan was weighed and poured into a beaker, 100 mL of UP water was added into the beaker, and mixed uniformly by stirring; 1 mL of glacial an acetic acid was added into the beaker and stirred until the chitosan was fully dissolved, and a chitosan solution was prepared.

5 g of PVA was added into the chitosan solution, stirred in a 90° C. water bath under heating until the PVA was completely dissolved, cooled and ultrasonically deformed, and a PVA/chitosan mixed solution with a mass ratio of 5:2 was obtained.

A genipin solution with 1% of mass fraction was prepared by dissolving the genipin in the UP water; and a vancomycin solution with 8% of mass fraction was prepared by dissolving the vancomycin in the UP water.

The chitosan, PVA, beaker, and stirrer were sterilized by ultraviolet irradiation, and the UP water was sterilized by autoclaving. The concentration of the genipin exceeded the required concentration for cross-linking to enhance anti-inflammatory effect.

The PVA/chitosan solution (a mass ratio of PVA to chitosan is 5:2) was added into a sterilized beaker, the vancomycin solution (each milliliter of hydrogel contains 8 mg of vancomycin) was added and stirred uniformly, followed by addition of the genipin solution (each milliliter of hydrogel contains 0.1 mg of genipin) and uniform stirring under dark condition to obtain the inner-layer hydrogel precursor solution.

### (3) Preparation of a Bilayer Bionic Drug-Loaded Hydrogel

The outer-layer PVA hydrogel was placed in a mould after removing excessive UP water, and 4 mL of the inner-layer hydrogel precursor solution was added into the mould and placed in a sterile and light-proof container for freezing and thawing at −18° C. for 1 h for three times, followed by standing for 3 days at room temperature to form an inner-layer hydrogel on the outer-layer PVA hydrogel, such that the bilayer bionic drug-loaded hydrogel was obtained.

The PVA in the inner-layer precursor solution and the PVA in the outer-layer hydrogel formed molecular linked by repeated freezing and thawing, and formed a first network by cross-linking in the inner-layer hydrogel. Meanwhile, the genipin made the chitosan form a second network by chemical cross-linking under the repeated freezing and thawing in the inner-layer hydrogel. A soft, moist, adherent, anti-inflammatory, haemostatic and pro-repair inner-layer hydrogel similar to subcutaneous tissue was obtained by adjusting cross-1 inking parameters of the double network system. The loading of vancomycin greatly improved the ability of hydrogel to resist infection by Gram-positive resistant bacteria and was suitable for the care of severely infected wounds such as war wounds.

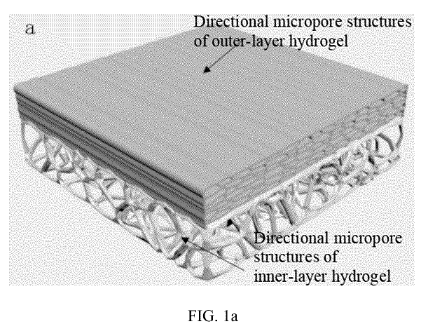

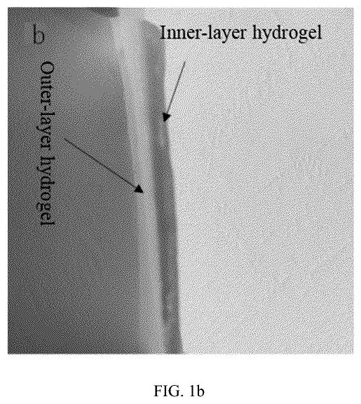

A structural diagram of a bilayer drug-loaded hydrogel of the present disclosure was shown in FIG. 1a and FIG. 1b. As shown in FIG. 1a, a structural diagram of the bilayer hydrogel indicated that the inner and outer layers had different microporous structures; as shown in FIG. 1b, an optical photograph of the bilayer hydrogel indicated that the inner and outer-layer hydrogels were tightly bonded together.

### Example 2

Provided herein was a method for preparing a bilayer bionic drug-loaded hydrogel, which included the following steps.

### (1) Preparation of an Outer-Layer Polyvinyl Alcohol (PVA) Hydrogel

5 g of PVA was weighed and poured into a beaker, 95 mL of ultrapure (UP) water was added into the beaker, fully dissolved in a 90° C. water bath under heating and stirring, cooled and ultrasonically deformed, and a PVA solution with a mass fraction of 5% was obtained.

Liquid nitrogen was added into a directional freezing device, and when the temperature of the directional freezing device was constant, a mould containing the PVA solution prepared was placed on the directional freezing device for directional freezing at −40° C. for 2 h; after the PVA solution was fully frozen, the mould was removed from the directional freezing device and demoulding was carried out; the demoulded PVA hydrogel was soaked in a sodium sulfate solution for 72 h, followed by soaking in the UP water for 48 h, and the UP water was changed every 4 h to remove salt ions from the PVA hydrogel, and the outer-layer PVA hydrogel was obtained.

The mechanical properties of the outer-layer PVA hydrogel were adjusted by adjusting the concentration of PVA and the concentration of salting-out liquid.

### (2) Preparation of an Inner-Layer Hydrogel Precursor Solution

2 g of chitosan was weighed and poured into a beaker, 100 mL of UP water was added into the beaker, and mixed uniformly by stirring; 1 mL of glacial an acetic acid was added into the beaker and stirred until the chitosan was fully dissolved, and a chitosan solution was prepared.

5 g of PVA was added into the chitosan solution, stirred in a 95° C. water bath under heating until the PVA was completely dissolved, cooled and ultrasonically deformed, and a PVA/chitosan mixed solution with a mass ratio of 5:2 was obtained.

A genipin solution with 1% of mass fraction was prepared by dissolving the genipin in the UP water; and a vancomycin solution with 8% of mass fraction was prepared by dissolving the vancomycin in the UP water.

The chitosan, PVA, beaker, and stirrer were sterilized by ultraviolet irradiation, and the UP water was sterilized by autoclaving. The concentration of the genipin exceeded the required concentration for cross-linking to enhance anti-inflammatory effect.

The PVA/chitosan solution (a mass ratio of PVA to chitosan is 5:2) was added into a sterilized beaker, the vancomycin solution (each milliliter of hydrogel contains 8 mg of vancomycin) was added and stirred uniformly, followed by addition of the genipin solution (each milliliter of hydrogel contains 0.1 mg of genipin) and uniform stirring under dark condition to obtain the inner-layer hydrogel precursor solution.

### (3) Preparation of a Bilayer Bionic Drug-Loaded Hydrogel

The outer-layer PVA hydrogel was placed in a mould after removing excessive UP water, and 4 mL of the inner-layer hydrogel precursor solution was added into the mould and placed in a sterile and light-proof container for freezing and thawing at −10° C. for 1.2 h for three times, followed by standing for 3 days at room temperature to form an inner-layer hydrogel on the outer-layer PVA hydrogel, such that the bilayer bionic drug-loaded hydrogel was obtained.

The PVA in the inner-layer precursor solution and the PVA in the outer-layer hydrogel formed molecular links by repeated freezing and thawing. And formed a first network by cross-linking in the inner-layer hydrogel. Meanwhile, the genipin made the chitosan form a second network by chemical cross-linking under the repeated freezing and thawing in the inner-layer hydrogel. A soft, moist, adherent, anti-inflammatory, haemostatic and pro-repair inner-layer hydrogel similar to subcutaneous tissue was obtained by adjusting cross-linking parameters of the double network system. The loading of vancomycin greatly improved the ability of hydrogel to resist infection by Gram-positive resistant bacteria and was suitable for the care of severely infected wounds such as war wounds.

### Comparative Example

### (1) Preparation of an Outer-Layer Polyvinyl Alcohol (PVA) Hydrogel

The preparation of the outer-layer PVA hydrogel was the same as that in Example 1.

### (2) Preparation of an Inner-Layer Polyvinyl Alcohol-Chitosan (PVA-CS) Hydrogel

An inner-layer hydrogel precursor solution was prepared according to the method provided in Example 1. Then the inner-layer hydrogel precursor solution was added into a mould and placed in a sterile and light-proof container for freezing and thawing at −18° C. for 1 h for three times, followed by standing to form the inner-layer hydrogel.

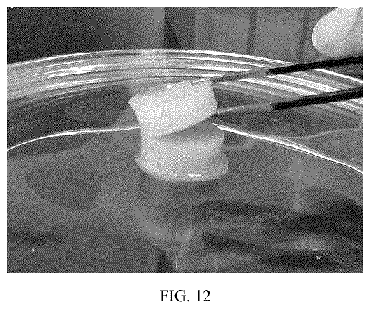

(3) The inner-layer hydrogel was putted on the outer-layer hydrogel, and added into the mould and placed in a sterile and light-proof container for freezing and thawing at −18° C. for 1 for three times, followed by standing for 3 days at room temperature. It was found that the inner-layer hydrogel was still separated from the outer-layer hydrogel, as shown in FIG. 12. This was because the inner-layer hydrogel and the outer-layer hydrogel each had formed its own cross-linking networks, the spatial mobility of the molecular chains of the inner-layer hydrogel and the outer-layer hydrogel was limited, and the cross-linking bonds of the inner-layer hydrogel and the outer-layer hydrogel were saturated, so that new cross-linking bonds between the inner-layer hydrogel and the outer-layer hydrogel could not be formed.

### Experimental Example 1 Microscopic Morphology

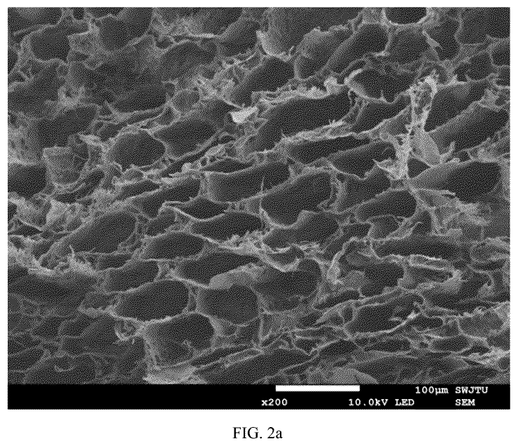

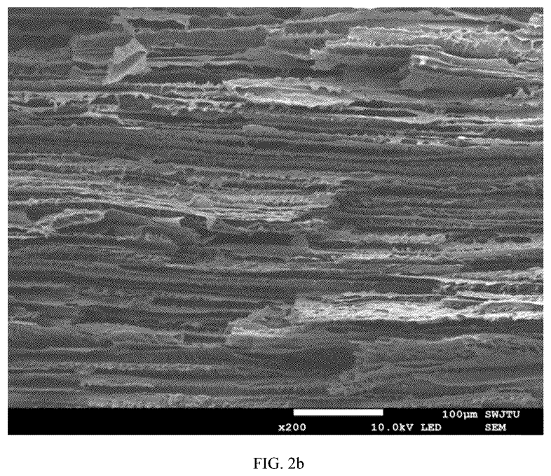

FIGS. 2a, 2b, 3 and 4 were microscopic morphology images of the bilayer bionic drug-loaded hydrogel prepared in Example 1.

The microscopic morphology of cross section a and longitudinal section b of the outer-layer hydrogel, as shown in FIG. 2a and FIG. 2b respectively, demonstrated that the outer-layer hydrogel had a directional microporous structure.

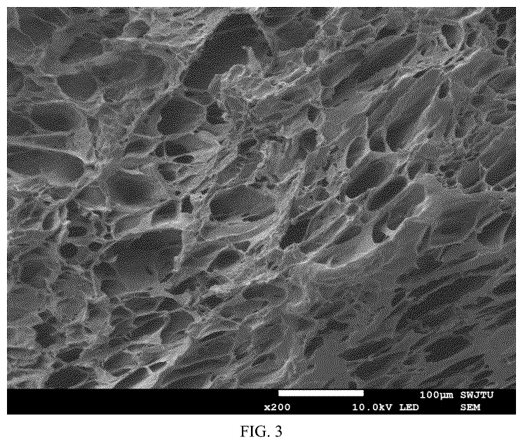

An image of a cross section of the inner-layer hydrogel under a scanning electron microscope, as shown in FIG. 3, indicated that an irregular porous structure of the inner-layer hydrogel provided a stronger water absorption property.

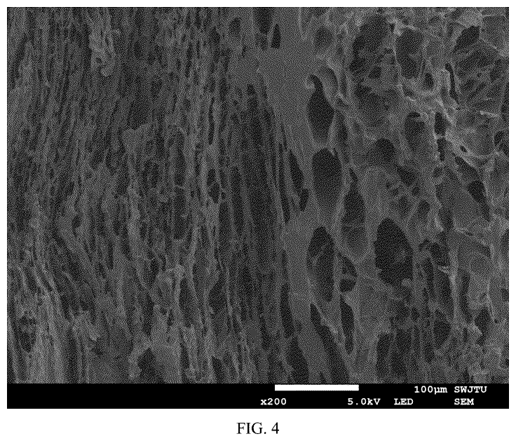

Images of a cross section and a longitudinal section of the bilayer hydrogel under a scanning electron microscope, as shown in FIG. 4, proved that the inner and outer layers of the bilayer hydrogel were tightly bonded together, and also proved significant structural differences between the outer and inner layers of the bilayer hydrogel as the outer layer had a directional microporous structure while the inner layer had an irregular porous structure.

### Experimental Example 2: Mechanical Properties

Tensile properties of materials were measured with a universal mechanical testing machine.

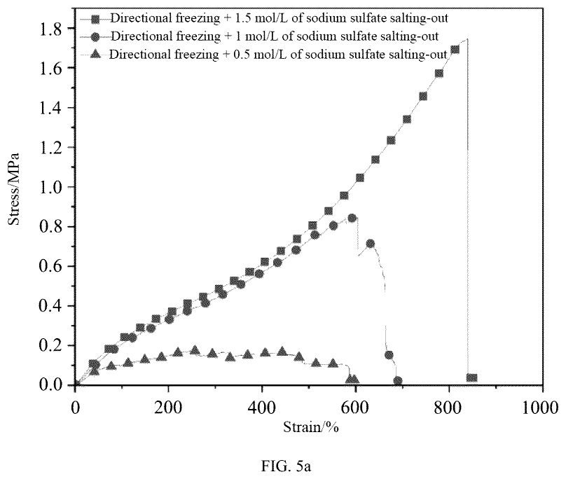

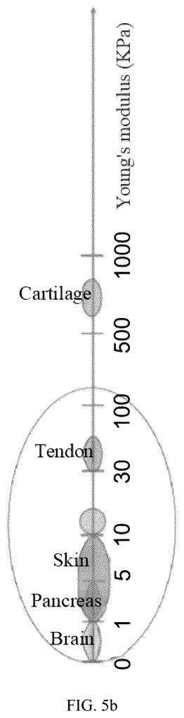

Mechanical properties of a hydrogel of the present disclosure were shown in FIG. 5a and FIG. 5b. FIG. 5a showed a stress-strain curve of an outer-layer hydrogel in tensile after removing salt ions by soaking in different concentrations of sodium sulfate solution for 4 days; and FIG. 5b showed a relationship between Young's modulus of hydrogel and Young's modulus of skin (the largest circle area represented a Young's modulus range of hydrogels). In FIG. 5a, an ultimate tensile strength of the outer-layer PVA hydrogel was 1.544±0.273 Mpa, a maximum elongation was 906.3441±53.9486%, and hydrogels with different mechanical properties could be prepared by adjusting concentrations of salt ions when performing salting-out. Therefore, the directional freezing and salting-out employed in the present disclosure had a synergistic effect. In FIG. 5b, the maximum Young's modulus of hydrogel was 170.3547 Kpa, and the adjustable range covered the Young's modulus of skin. The water content was about 75%, which was equivalent to the water content (71.77%) of skin. Therefore, the outer-layer hydrogel of the bilayer bionic drug-loaded hydrogel of the present disclosure had mechanical properties similar to those of skin and could withstand high intensity squeezing, pulling and rubbing, thereby protecting the wound of skin from negative external stimulation while keeping the wound surface dry and clean.

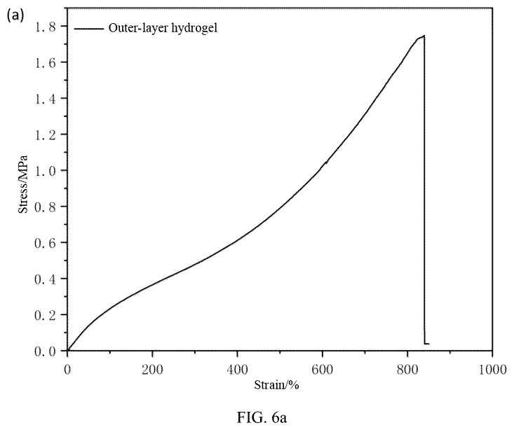

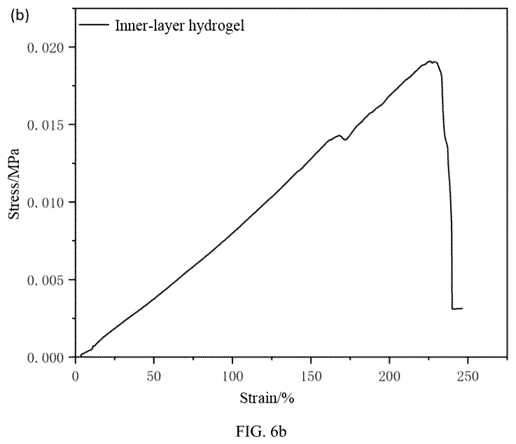

Mechanical properties of inner and outer hydrogels of the present disclosure were shown in FIG. 6a and FIG. 6b. As seen from FIG. 6a and FIG. 6b, the mechanical properties between the outer hydrogel and the inner hydrogel were different. As a structure determines properties, and the properties reflect the structure, the significant difference in structures between the outer and inner layer was verified again, which was consistent with the results of FIG. 4. Therefore, it was concluded from FIG. 6a and FIG. 6b that the directional microporous structure of the outer layer significantly improved the mechanical properties of hydrogel.

### Experimental Example 3: Infrared Spectroscopy

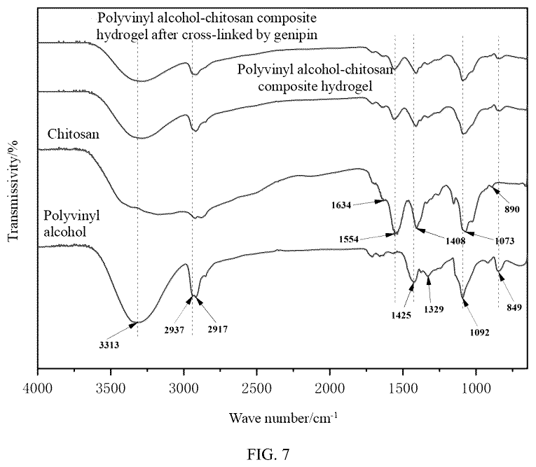

Materials are performed total reflection scanning using an infrared spectrometer. Infrared spectroscopy of an inner-layer hydrogel of the present disclosure is shown in FIG. 7. The infrared spectroscopy of the pure PVA hydrogel showed absorption peaks respectively caused by extensional vibration of oxhydryl (—OH) at 3200-3600 cm⁻¹, asymmetric and symmetrical extensional vibrations of alkyl (C—H) at 2937 and 2917 cm⁻¹, in-plane bending vibration of alkyl (C—H) at 1425 cm⁻¹, in-plane bending vibration of oxhydryl (—OH) at 1329 cm⁻¹, extensional vibration of C—O—C at 1092 cm⁻¹, and extensional vibration of C—C at 848 cm⁻¹. The infrared spectroscopy of the chitosan showed absorption peaks respectively caused by stretching of —C═O of peptide bond at 1634 cm⁻¹, bending of —NH of the peptide bond at 1554 cm⁻¹, and stretching of C—N of the peptide bond at 1408 cm⁻¹. A carbohydrate structure of the chitosan is at 1073 cm⁻¹, and pyranose ring of the chitosan is at 890 cm⁻¹. The infrared spectroscopy of CS/PVA composite hydrogel showed that characteristic peaks of the chitosan and the polyvinyl alcohol appear in the chitosan/PVA composite hydrogel. The infrared spectroscopy of the chitosan/PVA composite hydrogel after cross-linked by genipin was relatively stronger at 1634 cm⁻¹ compared to the infrared spectroscopy of the chitosan/PVA composite hydrogel, it was due to the formation of a large amount of amides after cross-linking between the chitosan and the genipin, which indicated that the chitosan and the genipin had a cross-linking reaction. In addition, the blue color of the composite hydrogel after the addition of genipin also proved that the genipin reacts with the chitosan.

### Experimental Example 4: Cytotoxicity

According to ISO 10993-5:1999 and GB/T 16886.5-2003, the biocompatibility of hydrogels with different drug-loading amounts was evaluated, wherein vancomycin (VCM)=x mg/ml represented the number of milligrams of vancomycin per milliliter hydrogel. Day 1: cell relative growth rates (RGR) in groups of VCM=0 mg/mL, VCM=2 mg/mL, VCM=5 mg/mL and VCM=8 mg/mL are 90.69%, 108.89%, 109.311% and 107.94% respectively. Day 3: the RGRs in groups of VCM=0 mg/mL, VCM=2 mg/mL, VCM=5 mg/mL and VCM=8 mg/mL are 74.23%, 83.13%, 99.27% and 95.68% respectively.

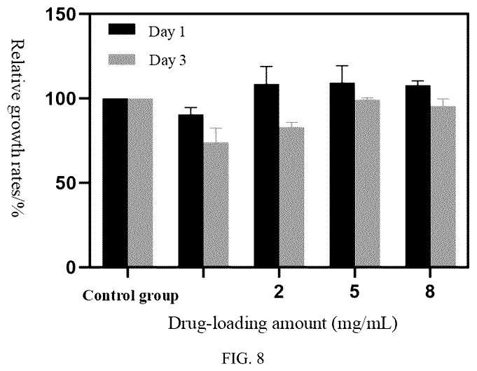

The cytocompatibility evaluation of the bilayer bionic drug-loaded hydrogel of the present disclosure was shown in FIG. 8. Combining results of FIG. 8 with the evaluation of the degree of cytotoxicity of samples according to rating criteria listed in Table 1, it was concluded that bilayer bionic drug-loaded hydrogel specimens of the present disclosure were rated as grade 1 and non-cytotoxic, and could be used as medical materials.

TABLE 1
Evaluation of RGR according to ISO10993-5:
1999 and GB/T 16886.5/12-2003

| Rating | RGR | Explanation |
|---|---|---|
| Grade 0 | ≥100 | Non-cytotoxic |
| Grade 1 | 75-99 | Non-cytotoxic |
| Grade 2 | 50-74 | May be cytotoxic |
| Grade 3 | 25-49 | Cytotoxic |
| Grade 4 | 1-25 | Cytotoxic |
| Grade 5 | 0 | Cytotoxic |

$$RGR ( \% ) = \frac{average absorbance values of experimental groups}{average absorbance values of negative experimental groups} \times 100.$$

### Experimental Example 5: Antioxidant Property

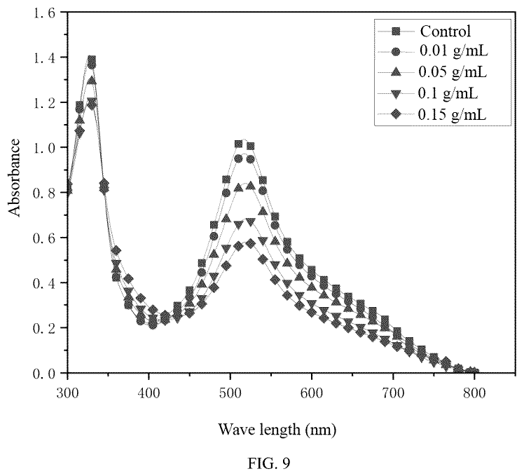

The antioxidant property of the hydrogel was evaluated using a DPPH radical scavenging method, the result was shown in FIG. 9. As shown in FIG. 9, with the increasing of hydrogel contents, an absorption peak at 517 nm of the hydrogel treatment group diminished, indicating that the hydrogel had a strong scavenging effect on DPPH radicals.

### Experimental Example 6: Antibacterial Property

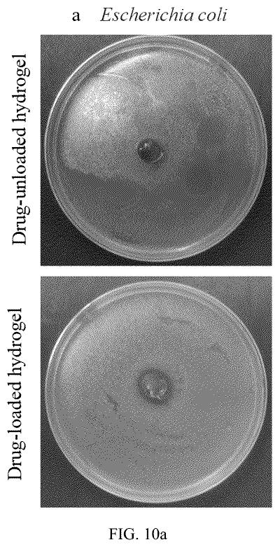

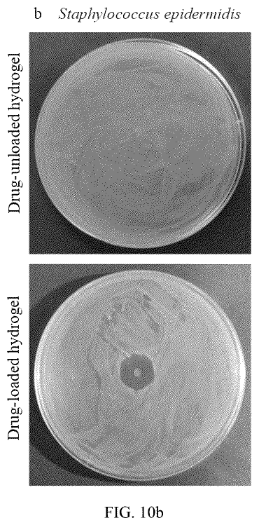

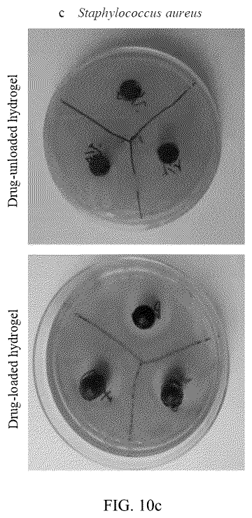

The antibacterial property of 5 mg/ml of hydrogel loading with vancomycin was evaluated using an inhibition zone method, as shown in FIG. 10a to FIG. 10c. FIG. 10a showed an antibacterial effect of the hydrogel on Escherichia coli; FIG. 10b showed an antibacterial effect of the hydrogel on Staphylococcus epidermidis; and FIG. 10c showed an antibacterial effect of the bilayer hydrogel on Staphylococcus aureus. According to results shown in FIG. 10a to FIG. 10c, a loaded-drug hydrogel group had a strong inhibitory effect on the Escherichia coli, Staphylococcus epidermidis and Staphylococcus aureus, while an unloaded-drug hydrogel had no obvious inhibitory effect. The weak antibacterial effect of the unloaded-drug hydrogel only relied on the component of the chitosan, and therefore inhibition effects on bacteria with higher concentrations were not obvious.

### Experimental Example 7: Adhesion Property

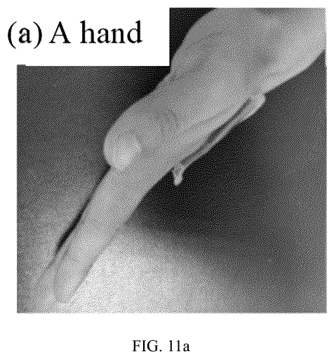

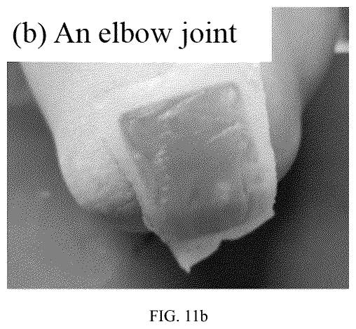

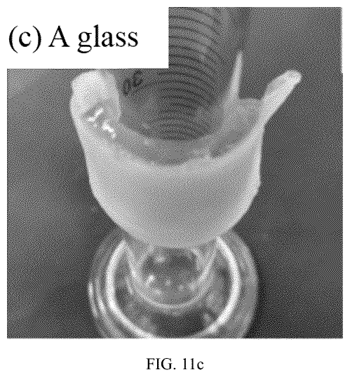

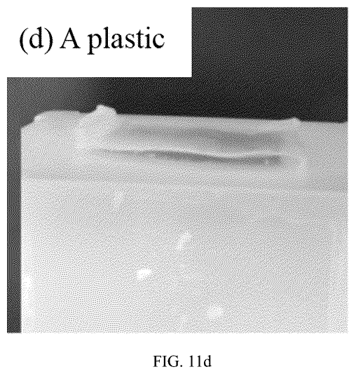

Adhesion effects of the hydrogel of the present disclosure on different materials surface were shown in FIG. 11a to FIG. 11d. FIG. 11a showed an adhesion of the hydrogel on hands; FIG. 11b showed an adhesion of the hydrogel on elbow joints;

FIG. 11c showed an adhesion of a bilayer hydrogel on glass; and FIG. 11d showed an adhesion of a bilayer hydrogel on plastic. As seen from FIG. 11a to FIG. 11d, the hydrogel had a good adhesion property, showing vertical adhesion to the back of hands, elbow joints, plastics and glass without falling off.

Although contents of the present disclosure have been described in detail with reference to the above preferred examples, it should be appreciated that the above description should not be considered as a limitation to the present disclosure. Various modifications and alternatives to the present disclosure will be apparent to those skilled in the art upon reading the foregoing. Accordingly, the scope of protection of the present disclosure should be defined by the attached claims.

What is claimed is:

1. A method for preparing a bilayer bionic drug-loaded hydrogel, comprising:
(S1) directionally freezing a polyvinyl alcohol aqueous solution, followed by soaking in a sodium sulfate solution and removal of salt ions to obtain an outer-layer hydrogel;
(S2) dissolving chitosan in water followed by addition of a pH adjuster to obtain a chitosan solution, and adding polyvinyl alcohol to the chitosan solution followed by heating under stirring to obtain a first mixture solution;
(S3) adding an antibacterial drug and genipin to the first mixture solution, followed by stirring in a dark environment to obtain a second mixture solution; and
(S4) spreading the second mixture solution on the outer-layer hydrogel, followed by freezing and thawing in a dark and clean environment to form an inner-layer hydrogel on the outer-layer hydrogel, such that the bilayer bionic drug-loaded hydrogel is obtained.

2. The method of claim 1, wherein in step (S1), a concentration of the sodium sulfate solution is 0.5-1.5 mol/L, and a weight percentage of the polyvinyl alcohol aqueous solution in the outer-layer hydrogel is 5-10%.

3. The method of claim 2, wherein the weight percentage of the polyvinyl alcohol aqueous solution in the outer-layer hydrogel is 5%.

4. The method of claim 1, wherein in step (S2), the pH adjuster is a weak acid.

5. The method of claim 4, wherein the pH adjuster is glacial acetic acid.

6. The method of claim 1, wherein in step (S2), a weight percentage of the polyvinyl alcohol in the first mixture solution is 5-10%, and a weight percentage of the chitosan in the first mixture solution is 2-4%.

7. The method of claim 6, wherein the chitosan and the polyvinyl alcohol are pre-sterilized by ultraviolet irradiation, and the water is pre-sterilized by autoclaving.

8. The method of claim 1, wherein in step (S2), the heating is performed at 85-95° C.

9. The method of claim 1, wherein in step (S3), the antibacterial drug is vancomycin, and a weight percentage of the genipin in the second mixture solution is 0.01-0.05%.

10. The method of claim 1, wherein in step (S4), the freezing and thawing is performed at −20-0° C. for 1-5 times, with a duration of 0.5-2 h for each time.

11. The method of claim 1, wherein the inner-layer hydrogel has a double-network structure.

12. The method of claim 1, wherein in step (S1), the freezing is performed at −80-0° C. for 0.5-4 h.
